(19) United States
(12) Patent Application Publication
(10) Pub. No.: US 2020/0339874 A1
(43) Pub. Date: Oct. 29, 2020

(54) BLUE EMITTER MATERIALS PRODUCED THROUGH NOVEL REDUCTIVE ELIMINATION PROCESS

(71) Applicant: R-Display & Lighting, LLC, Webster, NY (US)

(72) Inventor: Tommie L. Royster, Webster, NY (US)

(21) Appl. No.: 16/395,348

(22) Filed: Apr. 26, 2019

(51) Int. Cl. C09K 11/06; C07F 15/00; C07D 471/22; H01L 51/00

(52) U.S. Cl. CPC C09K 11/06; C07F 15/0093; C07D 471/22; H01L 51/5016; H01L 51/0087; C09K 2211/1018; H01L 51/0072

(57) ABSTRACT

Methods of producing compositions, which may be blue emitting compositions produced by reductive elimination of organometallic complexes with a central platinum group transition metal coordinated to a bridged tetradentate ligand comprised of chelating bis(azinyl)amine groups. The complexes may be heated under an inert atmosphere to produce a composition, which may be a deep blue and/or violet-blue emitting composition. Compositions that include one or more blue, for example, deep blue and/or violet-blue, emitting compound are also disclosed.

### BACKGROUND

### Technical Field

Light-emitting compositions of matter. In particular, compositions that are useful in organic light emitting diodes.

### Description of Related Art

Organic Light Emitting Diode (OLED) devices are based on strategic placement of organic thin films between electrodes (i.e. an anode and a cathode). Injection of holes and elections from the anode and cathode result in light emission through recombination of the holes and electrons in the light emitting layer of the “organic stack,” which is the set of layers between the anode and the cathode. The organic thin films in an OLED device are typically less than 50 nanometers (nm) in thickness, resulting in low voltage operations and potential to produce low power consuming devices. These attributes are advantageous in the use of OLED devices in image display and lighting applications.

The excited states generated from hole and electron injection setup two pathways for light emission. Singlet and Triplet exciton decay yield fluorescent and phosphorescent light respectively. The ratio of Singlet to Triplet exciton formation is 1:3. Therefore, emissive layers comprised of fluorescent dopant and host materials for harvesting singlet excitons have a theoretical limit of 25% for converting excitons into light. However, phosphorescent systems can theoretically convert 100% of the excitons generated into light by harvesting Singlet excitons (after intersystem conversion) and Triplet excitons. Emissive layers are comprised of a host material and a phosphorescent dopant.

The high efficiency of phosphorescent based OLED devices establishes a platform for manufacturing very low power consuming lighting and display applications. Based on the high efficiency, lower driving currents are required for light output, thereby establishing the potential for significant savings in power consumption. The shift from fluorescent based OLED devices to phosphorescent based devices in commercial applications has commenced. However, there are still problems that remain to be solved for broader application of phosphorescent based OLED devices to occur.

OLED displays have evolved from exclusive use of fluorescent emitter materials to incorporating more device efficient phosphorescent materials currently found in state-of-the art displays for smart phones and select HD-TV's. Specifically, red and green fluorescent emitters have been replaced with red and green phosphorescent emitters. A blue phosphorescent dopant has yet to be discovered that meets the industry requirements for color and device stability. This technology gap represents a major innovation opportunity for OLED emitter materials.

### SUMMARY

Organometallic complexes with a central platinum group transition metal coordinated to a bridged tetradentate ligand comprised of chelating bis(azinyl)amine groups were synthesized. A six-membered heteroatom ring is formed through coordination of the chelating ligand with the transition metal. An electron donating amine group serves as a bridge between the bis(azinyl)amine groups. The platinum group transition metal may be selected from the group consisting of platinum or palladium.

Heating the new class of organometallic materials within a horizontal vacuum sublimation system, produces novel reductive elimination products that are luminescent. Solutions of the reductive elimination products may produce blue (e.g., deep blue, violet-blue, or a combination thereof) light upon exposure to UV radiation.

In an example, a method of producing a composition, which may be a blue (e.g., deep blue, violet-blue, or a combination thereof) emitting composition, comprises: heating an organometallic compound with a central platinum group transition metal coordinated to a bridged tetradentate ligand comprised of chelating bis(azinyl)amine groups under an inert atmosphere, where the composition, which may be a blue (e.g., deep blue, violet-blue, or a combination thereof), is formed. The heating may be carried out at a temperature below the decomposition temperature of the organometallic compound. The heating may be carried out at 180° C. to 300° C., including all integer ° C. values and ranges therebetween. The heating may be carried out under a sub-ambient pressure in a flow of inert gas. The sub-ambient pressure may be 100 to 500 mTorr, including all integer mTorr values and ranges therebetween. The inert gas may be chosen from nitrogen, argon, and combinations thereof. The composition may comprise a an organic compound, which may be a blue (e.g., deep blue, violet-blue, or a combination thereof) emitting compound, having the following structure:

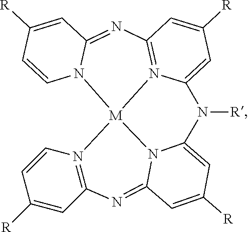

where R is selected from hydrogen, an alkyl group, or an aryl group; R′ is selected from an alkyl or aryl group and M is the central platinum group transition metal.

In an example, a composition comprises a compound, which may be a blue (e.g., deep blue, violet-blue, or a combination thereof) emitting compound, that may have the following structure:

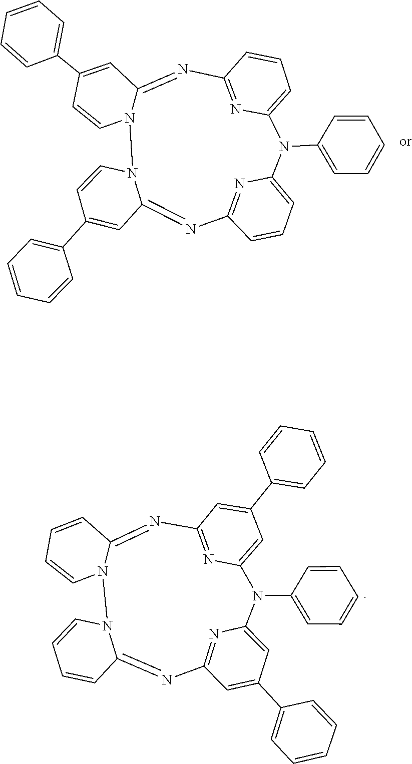 [SMILES: C1=CN2/C(=N\c3cccc(n3)N(c3ccccc3)c3cccc(n3)/N=C3/C=C(c4ccccc4)C=CN32)C=C1c1ccccc1 ; C1=C/C2=N/c3cc(-c4ccccc4)cc(n3)N(c3ccccc3)c3cc(-c4ccccc4)cc(n3)/N=C3/C=CC=CN3N2C=C1]

The composition and/or a compound may exhibit an emission maximum in the range of 390 to 490 nm, including all integer nm values and ranges therebetween. The composition may comprise a material made by a method of the present disclosure.

### BRIEF DESCRIPTION OF THE DRAWINGS

For a general understanding of the present invention, reference is made to the drawings. The drawings are to be considered as depicting exemplary embodiments of the invention, and not to be considered as limiting the invention solely to the embodiments depicted.

FIG. 1 is an illustration of the general chemical structure of organometallic material used in the process.

FIG. 2 is an illustration of the chemical structure of a first light-emitting composition of produced form the process.

FIG. 3 is an illustration of the chemical structure of a second light-emitting composition produced from the process.

FIG. 4 is a photoluminescence spectrum of emitter material isolated from the reduction elimination process represented in FIG. 3.

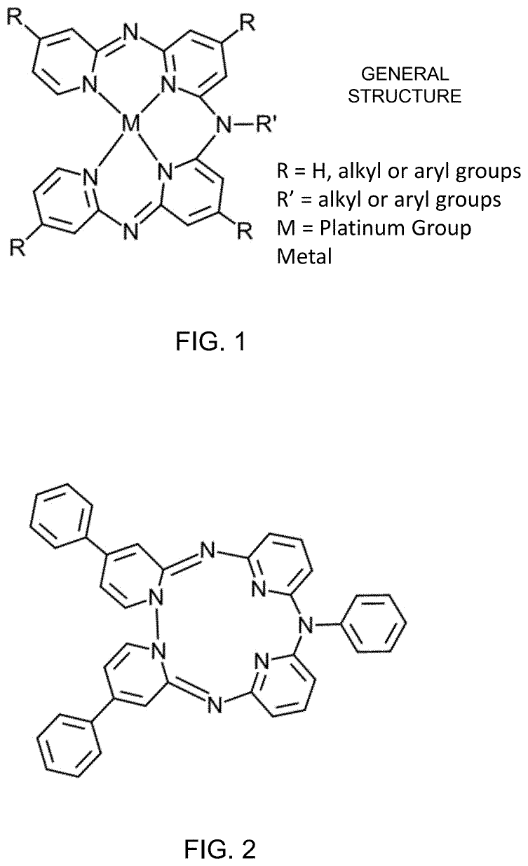

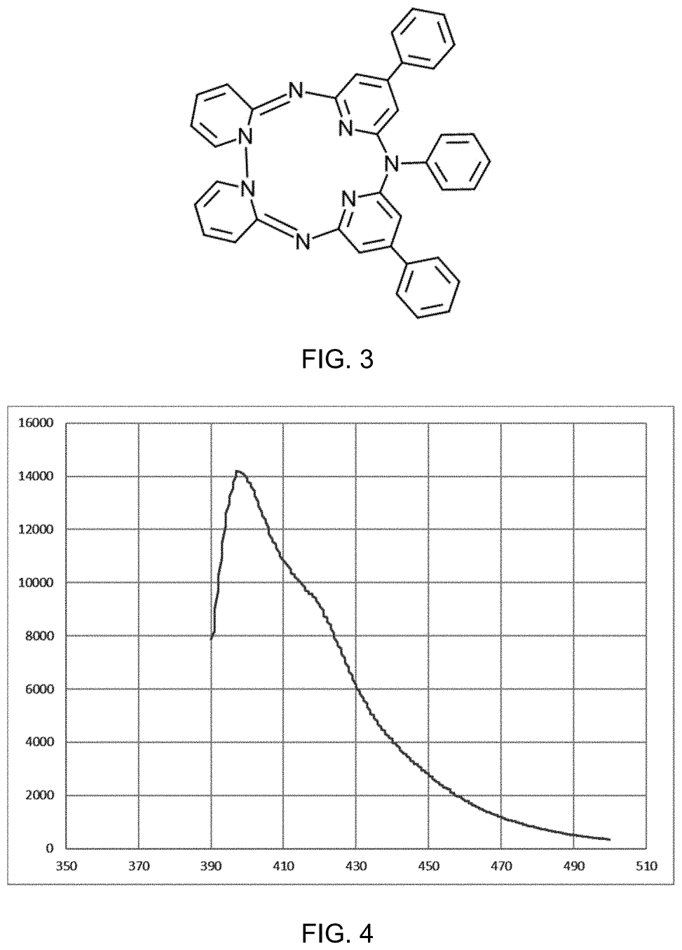

### DETAILED DESCRIPTION

The present invention will be described in connection with certain preferred embodiments. However, it is to be understood that there is no intent to limit the invention to the embodiments described. On the contrary, the intent is to cover all alternatives, modifications, and equivalents as may be included within the spirit and scope of the invention as defined by the appended claims.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). Illustrative examples of groups include:

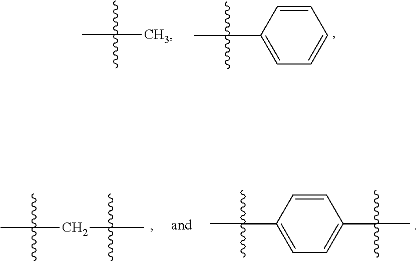

As used herein, unless otherwise indicated, the term “alkyl group” refers to branched or unbranched saturated hydrocarbon groups. Examples of alkyl groups include, but are not limited to, methyl groups, ethyl groups, propyl groups, butyl groups, isopropyl groups, tert-butyl groups, and the like. For example, the alkyl group can be a C₁ to C₁₂, including all integer numbers of carbons and ranges of numbers of carbons therebetween, alkyl group. The alkyl group can be unsubstituted or substituted with one or more substituent. Examples of substituents include, but are not limited to, various substituents such as, for example, aliphatic groups (e.g., alkyl groups, alkenyl groups, and alkynl groups), aryl groups, alkoxide groups, carboxylate groups, carboxylic acids, ether groups, amine groups, and the like, and combinations thereof.

As used herein, unless otherwise indicated, the term “aryl group” refers to C₅ to C₁₂, including all integer numbers of carbons and ranges of numbers of carbons therebetween, aromatic or partially aromatic carbocyclic groups. An aryl group can also be referred to as an aromatic group. The aryl groups can comprise polyaryl groups such as, for example, fused ring or biaryl groups. The aryl group can be unsubstituted or substituted with one or more substituent. Examples of substituents include, but are not limited to, substituents such as, for example, aliphatic groups (e.g., alkenes, alkynes, and the like), aryl groups, alkoxides, carboxylates, carboxylic acids, ether groups, and the like, and combinations thereof. Examples of aryl groups include, but are not limited to, phenyl groups, biaryl groups (e.g., biphenyl groups and the like), and fused ring groups (e.g., naphthyl groups and the like).

Through a combination of synthetic work, evaluation of sublimation properties and photoluminescence studies, the Applicant has discovered a novel reductive elimination process of organometallic compositions that produces luminescent materials. Heating the novel organometallic compounds contained in a quartz boat within a horizontal sublimation tube, resulted in the compound going through a melting phase prior to a reductive elimination process. The volatile elimination product condensed within a lower temperature zone of the sublimation tube. Solutions of the elimination product may produce blue (e.g., deep blue, violet-blue, or a combination thereof) light when exposed to UV radiation. The following schematic representation illustrates the novel reductive elimination process. The general structure of the organometallic used in the process is shown in FIG. 5. Structures of the resulting luminescent compositions are represented in FIGS. 6 and FIG. 7. An Example of a photoluminescence spectrum produced from the reduction elimination product is represented in FIG. 8.

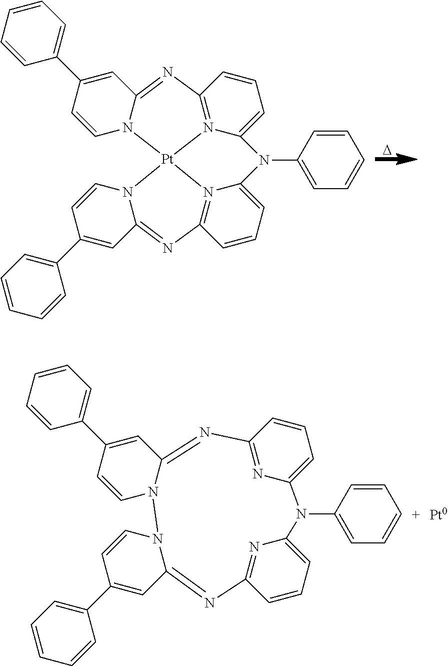

The present disclosure provides processes that may be used to produce reductive elimination products from organometallic complexes with a central platinum group transition metal coordinated to a bridged tetradentate ligand comprised of chelating bis(azinyl)amine groups. Non-limiting examples of organometallic complexes include:

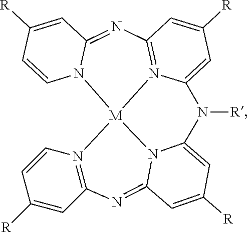

where R is independently at each occurrence chosen from hydrogen, alkyl groups (e.g., alkyl groups as described herein), and aryl groups (e.g. aryl groups as disclosed herein); and R′ is chosen from selected from alkyl groups (e.g., alkyl groups as described herein), and aryl groups (e.g. aryl groups as disclosed herein) and M is the central platinum group transition metal (e.g., Pt, Ni, and Pd).

The present disclosure also provides compositions comprising one or more materials produced from the process of the present disclosure. In non-limiting examples, a composition comprises a compound, which may be a blue (e.g., deep blue, violet-blue, or a combination thereof) emitting compound, having the following structure:

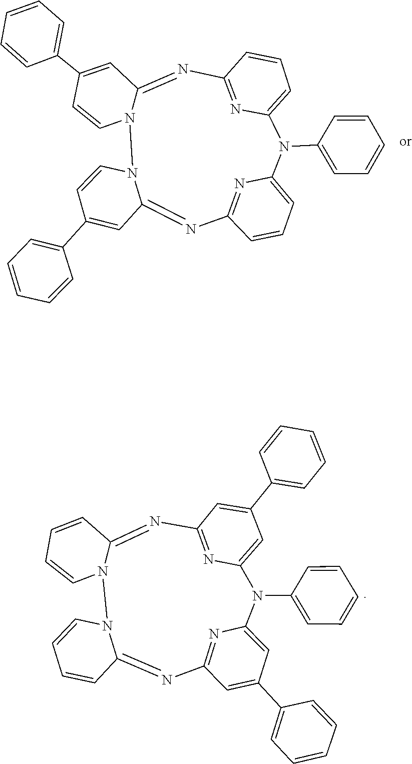 [SMILES: C1=CN2/C(=N\c3cccc(n3)N(c3ccccc3)c3cccc(n3)/N=C3/C=C(c4ccccc4)C=CN32)C=C1c1ccccc1 ; C1=C/C2=N/c3cc(-c4ccccc4)cc(n3)N(c3ccccc3)c3cc(-c4ccccc4)cc(n3)/N=C3/C=CC=CN3N2C=C1]

The steps of the methods described in the various embodiments and examples disclosed herein are sufficient to carry out the methods of the present disclosure. Thus, in an embodiment, a method consists essentially of a combination of the steps of the methods disclosed herein. In another embodiment, a method consists of such steps.

### EXAMPLE 1

### Synthesis of 2-{N-Phenyl[4-phenyl-6-(2-pyridylamino)-2-pyridyl]amino}-4-phenyl-6-(2-pyridylamino)pyridine (Ligand Used to Organometallic Synthesis)

Step 1. Working in a drybox, the reactant 2,6-dichloro-4-phenylpyridine (25 g, 111.7 mmol) was added to a 250 mL Schlenk flask with 70 mL of Toluene. The solution was stirred dissolving the bulk of the white solid. The reagent sodium tert-butoxide (10.5 g, 109 mmol) was then added followed by catalyst bis(diphenylphosphino)ferrocene]dichloropalladium(II)-*CH₂Cl₂. (1.1 g, 1.3 mmol). An additional 20 mL of Toluene was used to wash reagents into the flask. With stirring, the reactant aniline (3.7 mL, 40 mmol) was then added slowly. The sealed reaction flask was placed in a heated oil bath with the temperature set at approximately 100 C.

After greater than 48 hours, the heat was removed and additional Toluene was (75-100 mL) was added to the still warm dark brown solution. After stirring the solution was filtered (very little solid was collected on the medium porosity glass frit). The solvent was removed using a rotary evaporator. The product was washed into another flask before and the remaining solvent removed. The flask was connected to a bent glass elbow and Schlenk flask. The flask was heated under vacuum while cooling the Schlenk flask with a acetone/ice bath. The bath temperature was adjusted above 130 C to collect volatile excess starting material and impurities

Step 2. Working in the drybox, the following reactants were added to the flask containing the intermediate product isolated from step 1:
1. 2-aminopyridine (2 mol per mol of initial aniline)
2. Sodium t-butoxide (1.4 mol relative to mol of aminopyridine)
3. Pd catalyst. (1.3 mol per mol of aminopyridine)
4. 100 mL Toluene.

The reaction mixture was heated above 120 C for two days. The product was isolated after filtration followed by solvent removal. The final product was purified by sublimation.

### EXAMPLE 2

### General Method of Pt Complex Preparation

The following description is provided as an example of a general method of the steps of coordinating a central platinum group transition metal to a bridged tetradentate ligand to form the phosphorescent emitter materials of the present disclosure.

In a general reaction, 3 mmol of the Pt complex K₂PtCl₄ was weighed out and transferred to a reaction flask. High purity water (8 mL) was then added to the flask and the solution stirred to dissolve the Pt salt. While stirring, 30 mL of methanol was added followed by the addition of the solid bis(azinyl)amine chelating ligand (6 mmol). An additional 5 mL of the solvent was used to rinse down any remaining ligand. After purging the flask with nitrogen, the flask was sealed with a Rodavise cap (a condenser with a nitrogen bubbler can also be used).

The reaction was heated (75-80° C.) in an oil bath. After 10-16 hours, the heat was removed and the reaction flask allowed to cool to room temperature. Additional methanol and water was added (60 mL and 20 mL respectively). While stirring, 20 mL of an aqueous solution with excess KOH was added dropwise. The solution was again purged with nitrogen before stirring for 2-3 hours with low heat. The solvent volume was reduced before introducing additional water. The product was allowed to settle before collecting by filtration. The product was confirmed by LC-MS analysis to be the composition shown in FIG. 2, before purification by sublimation.

### EXAMPLE 3

The following general procedure was used to promote the reductive elimination process of novel organometallic compounds. The organometallic material was placed in a quartz boat that was transferred to a 1 inch sublimation tube contained within a tube furnace. The sublimation tube was connected to vacuum pump capable of achieving a vacuum of 10⁻³ Torr. The other end of the sublimation tube was connected to a nitrogen inlet valve. With the nitrogen inlet valve closed, the sublimation tube was evacuated until a vacuum of at least 50 mTorr was achieved. The nitrogen inlet valve was then opened and adjusted to achieve a vacuum in the range of 200 mTorr to 400 mTorr with nitrogen serving as a carrier gas. The temperature of the furnace was slowing increased resulting in the material melting above 150° C. Volatile material was observed condensing within the lower temperature zone of the sublimation tube set above 200° C. The temperature was increased until a temperature above 280° C. was reached. The material condensed on the walls of the sublimation tube was then fully characterized.

The foregoing description of technology and the invention is merely exemplary in nature of the subject matter, manufacture, and use of the invention and is not intended to limit the scope, application, or uses of any specific invention claimed in this application or in such other applications as may be filed claiming priority to this application, or patents issuing therefrom. The following definitions and non-limiting guidelines must be considered in reviewing the description.

The headings in this disclosure (such as “Background” and “Summary”) and sub-headings used herein are intended only for general organization of topics within the present technology, and are not intended to limit the disclosure of the present technology or any aspect thereof. In particular, subject matter disclosed in the “Background” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

To the extent that other references may contain similar information in the Background herein, said statements do not constitute an admission that those references are prior art or have any relevance to the patentability of the technology disclosed herein. Any discussion in the Background is intended merely to provide a general summary of assertions.

The description and specific examples, while indicating embodiments of the technology disclosed herein, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

To the extent employed herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As referred to herein, all compositional percentages are by weight of the total composition, unless otherwise specified. As used herein, the words “comprise,” “include,” contain,” and variants thereof are intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Having thus described the basic concept of the disclosure, it will be apparent to those skilled in the art that the foregoing detailed disclosure is intended to be presented by way of example only and is not limiting. Various alterations, improvements, and modifications will occur to those skilled in the art, though not expressly stated herein. These alterations, Improvements, and modifications are intended to be suggested hereby, and are within the spirit and scope of the disclosure. Additionally, the recited order of processing elements or sequences, or the use of numbers, letters, or other designations therefore, is not intended to limit the claimed processes to any order except as may be expressly stated in the claims.

1. A method of producing a blue emitting composition of claim 8 comprising:
heating an organometallic compound with a central platinum group transition metal coordinated to a bridged tetradentate ligand comprised of chelating bis(azinyl)amine groups under an inert atmosphere, wherein the blue emitting composition is formed.

2. The method of claim 1, wherein the heating is carried out at a temperature below the decomposition temperature of the organometallic compound.

3. The method of claim 1, wherein the heating is carried out at 180° C. to 300° C.

4. The method of claim 1, wherein the heating is carried out under a sub-ambient pressure in a flow of inert gas.

5. The method of claim 4, wherein the sub-ambient pressure is 100 to 500 mTorr.

6. The method of claim 4, wherein the inert gas is chosen from nitrogen, argon, and combinations thereof.

7. The method of claim 1, wherein the organometallic compound has the following structure: 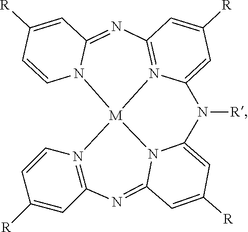
wherein R is selected from hydrogen, an alkyl group, or an aryl group; R′ is selected from an alkyl or aryl group and M is the central platinum group transition metal.

8. A composition comprising a compound having the following structure:

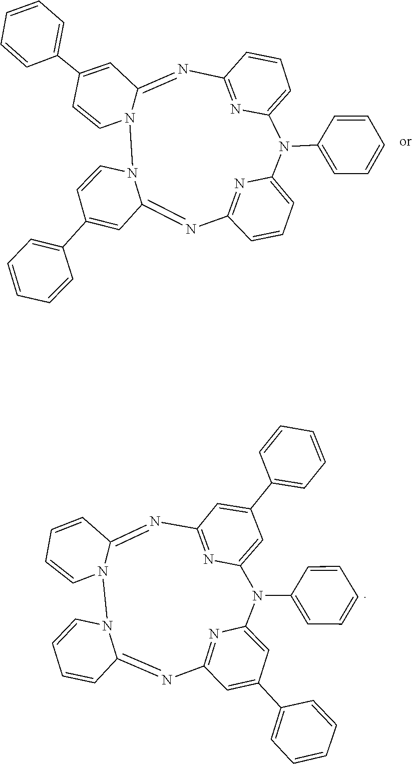 [SMILES: C1=CN2/C(=N\c3cccc(n3)N(c3ccccc3)c3cccc(n3)/N=C3/C=C(c4ccccc4)C=CN32)C=C1c1ccccc1 ; C1=C/C2=N/c3cc(-c4ccccc4)cc(n3)N(c3ccccc3)c3cc(-c4ccccc4)cc(n3)/N=C3/C=CC=CN3N2C=C1]

9. The composition of claim 8, wherein the composition exhibits an emission maximum in the range of 390 to 490 nm.

10. A composition comprising a material made by a method of claim 1.

11. The composition of claim 10, wherein the composition exhibits an emission maximum in the range of 390 to 490 nm.

12. The composition of claim 10, wherein the composition comprises a compound having the following structure: 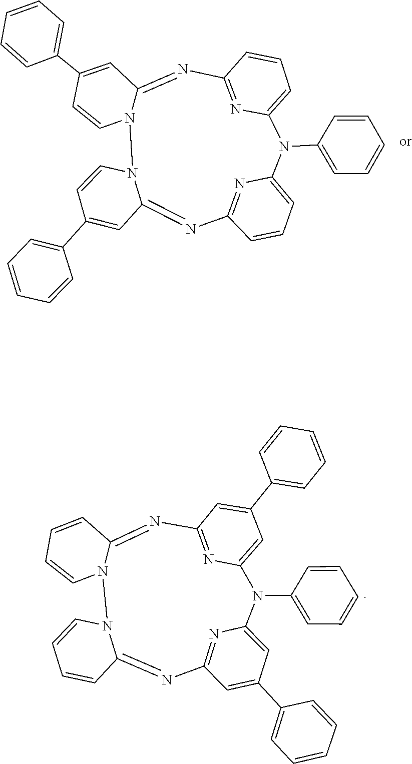 [SMILES: C1=CN2/C(=N\c3cccc(n3)N(c3ccccc3)c3cccc(n3)/N=C3/C=C(c4ccccc4)C=CN32)C=C1c1ccccc1 ; C1=C/C2=N/c3cc(-c4ccccc4)cc(n3)N(c3ccccc3)c3cc(-c4ccccc4)cc(n3)/N=C3/C=CC=CN3N2C=C1]
